(19) United States
(12) Patent Application Publication
(10) Pub. No.: US 2024/0176603 A1
(43) Pub. Date: May 30, 2024

(54) Software Call Translations for On-Device Machine Learning Execution

(71) Applicant: Meta Platforms, Inc., Menlo Park, CA (US)

(72) Inventors: Roman Georg RAEDLE, Palm Desert, CA (US); Christopher Robert Harper KLAIBER, Palo Alto, CA (US); Yinglao LIU, Sunnyvale, CA (US); Shiyong FANG, Fremont, CA (US); Pranav DESHPANDE, Frisco, TX (US); Hung Shek NGAN, Kirkland, WA (US); Anoop Kumar SINHA, Palo Alto, CA (US)

(21) Appl. No.: 18/059,613

(22) Filed: Nov. 29, 2022

(51) Int. Cl. G06F 8/51; G06F 8/36

(52) U.S. Cl. CPC G06F 8/51; G06F 8/36

(57) ABSTRACT

Aspects of the present disclosure are directed to translating application calls for on-device machine learning execution. A translation layer supports on-device machine learning execution by translating JavaScript software application call data to achieve interoperability with on-device machine learning models. For example, JavaScript software applications interact with data, such as images, audio, video, and/or text, in a format or data type that is compatible with the application. On the other hand, machine learning models interact with data in a form conducive to mathematical operations, such as a data structure representation (e.g., tensor representation). Implementations translate data types and/or data files to provide compatible data to each of a native JavaScript software application and on-device machine learning models. The translation layer can translate JavaScript application calls to provide compatible data to the machine learning model(s), and output from the machine learning model(s) to provide compatible data to the JavaScript application.

### TECHNICAL FIELD

The present disclosure is directed to translating application calls for on-device machine learning execution.

### BACKGROUND

Machine learning models have grown popular due to their unique and diverse functionality. These models can augment images or videos, track objects, perform natural langue processing functions, generate a range of computer predictions, and perform several other types of impactful software functionality. Conventional machine learning models are deployed at a server, in the cloud, or at another suitable computing device with ample quantities of computing resources and sophisticated software environments. Often, computing devices with less sophisticated computing environments and/or less available computing resources are not able to support application functionality that includes machine learning model execution.

### BRIEF DESCRIPTION OF THE DRAWINGS

FIG. 1 is a block diagram illustrating an overview of devices on which some implementations can operate.

FIG. 2 is a block diagram illustrating an overview of an environment in which some implementations can operate.

FIG. 3 is a block diagram illustrating components which, in some implementations, can be used in a system employing the disclosed technology.

FIGS. 4A, 4B an d 4C are conceptual diagrams of software architecture that translates application calls for on-device machine learning execution.

FIG. 5 is a conceptual diagram of data translations between a native application and machine learning model(s).

FIG. 6 is a flow diagram illustrating a process used in some implementations for translating application calls for on-device machine learning execution.

The techniques introduced here may be better understood by referring to the following Detailed Description in conjunction with the accompanying drawings, in which like reference numerals indicate identical or functionally similar elements.

### DETAILED DESCRIPTION

Aspects of the present disclosure are directed to translating application calls for on-device machine learning execution. Implementations support on-device machine learning execution using a translation layer that translates JavaScript software application call data to achieve interoperability with on-device machine learning models. The on-device machine learning execution refers to machine learning performed on a mobile device, client device, edge device, or any other suitable device that is not located in the cloud. Conventional machine learning is often performed at a server or other suitable cloud device that includes sophisticated software environments and/or large quantities of available computing resources. Machine learning in the context of constrained computing resources and less sophisticated software environments presents unique challenges.

Implementations leverage on-device runtime environments, efficient machine learning models, and a translation layer to provide machine learning functionality to JavaScript applications running natively on-device. Software applications running natively on a device often interact with data formatted in a manner that is conducive to the software application. For example, a React Native JavaScript application can comprise data types for audio, video, images, and text that support interactions with the application, such as playing the audio, display of the video, and/or display of the image. Some object data types also refer to underlying data files, such as image files, video files, audio files, text files, and the like. These data files are also stored in file formats that are conducive to JavaScript application functionality.

Example file formats compatible with JavaScript application functionality include Bitmap, Portable network graphics (PNG), Graphics Interchange Format (GIF), Joint Photographic Experts Group (JPEG), Moving Picture Experts Group (MPEG), Windows media video (WMV), MP4, MP3, Windows Media Audio (WMA), Waveform audio file (WAV), any suitable text data file, or any other suitable data file that conventionally interacts with JavaScript applications. However, these data types and/or file formats are often incompatible with machine learning execution. For example, machine learning models may interact with the same type of data in a tensor representation.

A tensor is a data structure that stores data in a manner that is conducive for manipulation and/or calculation, such as a multi-dimensional array or matrix. For example, libraries, models, and other software structures can be configured to efficiently perform manipulations and calculations on tensors. Data files, such as images, video, audio, text, and other data, are often stored in a file format that does not comprise a tensor representation of the data, for example to support efficient interactions with JavaScript software applications. However, machine learning model execution often involves a high number of calculations on input data, and these data files are not compatible with this type of machine learning model interaction. Implementations of a translation layer can convert these data file into tensor representations that are compatible with machine learning model execution. In some implementations, the translation layer can translate any suitable data type (e.g., media data types, text data type, etc.) or data format compatible with a native JavaScript software application into a data type or data format that complies with the compatibility of a loaded machine learning model.

Output from machine learning model(s) is also often in a format that is compatible with the machine learning model(s), such as a tensor representation, but incompatible with a native JavaScript software application. For example, software application functionality, such as displaying an image/video file or playing an audio file, is often inefficient or otherwise impractical using tensor representations of data. Implementations of the translation layer can also convert tensor representations of data into conventional data types (e.g., image, audio, video, or text data types) and/or data formats that are compatible with software application functionality. Accordingly, the translation layer can support machine learning model functionality alongside JavaScript application functionality by providing each component compatible data.

In some implementations, the translation layer may perform data introspection on JavaScript application call data and/or data output from the machine learning model(s). For example, JavaScript application call data can include a pointer to a data blob, such as an unidentified data file in memory, or any other suitable unidentified data. The translation layer can perform introspection of the call data to support translation to on-device runtime environment/machine learning model compatible data representations, such as C++ data types and/or tensor representations. Implementations can introspect the data types or data files of the call data using one or more introspection methods/functions that test a variety of data types/files (e.g., functions to test whether data is an image, audio, video, text file, etc.). For example, one of the method calls can return a positive value that indicates the data type(s) and/or type(s) of data file(s) (e.g., image, audio, video, text file, etc.) for the call data. The translation layer can then perform the relevant transformations for the introspected data types and/or data files to translate the call data to data compatible with the on-device runtime environment/machine learning model(s).

In some implementations, the translation layer may also perform data introspection on the data output by machine learning model(s) to introspect the data represented by the tensor representation. For example, data output by machine learning model(s) can represent an image, video, audio, text, or any other suitable data. The translation layer can perform introspection on the output data to support translation to JavaScript application compatible data types and/or data file formats. Implementations can introspect the data represented by the output data using one or more introspection methods/functions that test a variety of data types/formats (e.g., functions to test whether the tensor representation is an image, audio, video, text file, etc.). For example, one of the method calls can return a positive value that indicates the data represented by the output data. The translation layer can then perform the relevant transformations for the introspected data types and/or data formats to translate the output data into JavaScript application compatible data. The calling JavaScript application can perform software functions with the JavaScript application compatible data that is returned in response to the software call. These functions can include displaying an image or video, playing an audio file, processing and/or displaying a text file, or any other suitable software functions.

Conventional machine learning is performed on a cloud device with ample computing resources and sophisticated environments. These implementations fail to scale to less sophisticated environments with limited resources. Existing on-device machine learning functionality for mobile devices provides a limited experience, with limitations on machine learning model types, functionality, and flexibility. For example, conventional on-device machine learning frameworks for mobile devices do not provide Native JavaScript applications access to machine learning models.

Implementations disclosed herein provide a translation layer that translates data types and data formats to support a greater variety of on-device machine learning models and a great diversity of functionality. For example, images, videos, audio, and/or text data can be transformed to tensor representation for on-device machine learning model inference. Some implementations also include data introspection to determine the relevant transformations to perform. Implementations provide JSI-compatible native applications to use JavaScript for on-device machine learning model inference using multi-directional software call and data translation so that both the JavaScript applications and the machine learning models are provided with compatible data.

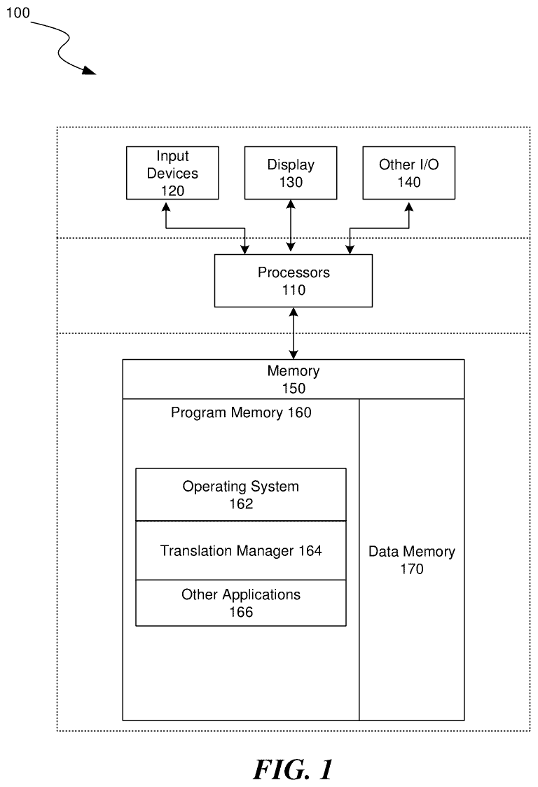

Several implementations are discussed below in more detail in reference to the figures. FIG. 1 is a block diagram illustrating an overview of devices on which some implementations of the disclosed technology can operate. The devices can comprise hardware components of a device 100 that translate application calls for on-device machine learning execution. Device 100 can include one or more input devices 120 that provide input to the Processor(s) 110 (e.g., CPU(s), GPU(s), HPU(s), etc.), notifying it of actions. The actions can be mediated by a hardware controller that interprets the signals received from the input device and communicates the information to the processors 110 using a communication protocol. Input devices 120 include, for example, a mouse, a keyboard, a touchscreen, an infrared sensor, a touchpad, a wearable input device, a camera- or image-based input device, a microphone, or other user input devices.

Processors 110 can be a single processing unit or multiple processing units in a device or distributed across multiple devices. Processors 110 can be coupled to other hardware devices, for example, with the use of a bus, such as a PCI bus or SCSI bus. The processors 110 can communicate with a hardware controller for devices, such as for a display 130. Display 130 can be used to display text and graphics. In some implementations, display 130 provides graphical and textual visual feedback to a user. In some implementations, display 130 includes the input device as part of the display, such as when the input device is a touchscreen or is equipped with an eye direction monitoring system. In some implementations, the display is separate from the input device. Examples of display devices are: an LCD display screen, an LED display screen, a projected, holographic, or augmented reality display (such as a heads-up display device or a head-mounted device), and so on. Other I/O devices 140 can also be coupled to the processor, such as a network card, video card, audio card, USB, firewire or other external device, camera, printer, speakers, CD-ROM drive, DVD drive, disk drive, or Blu-Ray device.

In some implementations, the device 100 also includes a communication device capable of communicating wirelessly or wire-based with a network node. The communication device can communicate with another device or a server through a network using, for example, TCP/IP protocols. Device 100 can utilize the communication device to distribute operations across multiple network devices.

The processors 110 can have access to a memory 150 in a device or distributed across multiple devices. A memory includes one or more of various hardware devices for volatile and non-volatile storage, and can include both read-only and writable memory. For example, a memory can comprise random access memory (RAM), various caches, CPU registers, read-only memory (ROM), and writable non-volatile memory, such as flash memory, hard drives, floppy disks, CDs, DVDs, magnetic storage devices, tape drives, and so forth. A memory is not a propagating signal divorced from underlying hardware; a memory is thus non-transitory. Memory 150 can include program memory 160 that stores programs and software, such as an operating system 162, translation manager 164, and other application programs 166. Memory 150 can also include data memory 170, e.g., images, video, audio, or other suitable application call data, parameters for machine learning model(s), tensor representations of data, configuration data, settings, user options or preferences, etc., which can be provided to the program memory 160 or any element of the device 100.

Some implementations can be operational with numerous other computing system environments or configurations. Examples of computing systems, environments, and/or configurations that may be suitable for use with the technology include, but are not limited to, personal computers, server computers, handheld or laptop devices, cellular telephones, wearable electronics, gaming consoles, tablet devices, multiprocessor systems, microprocessor-based systems, set-top boxes, programmable consumer electronics, network PCs, minicomputers, mainframe computers, distributed computing environments that include any of the above systems or devices, or the like.

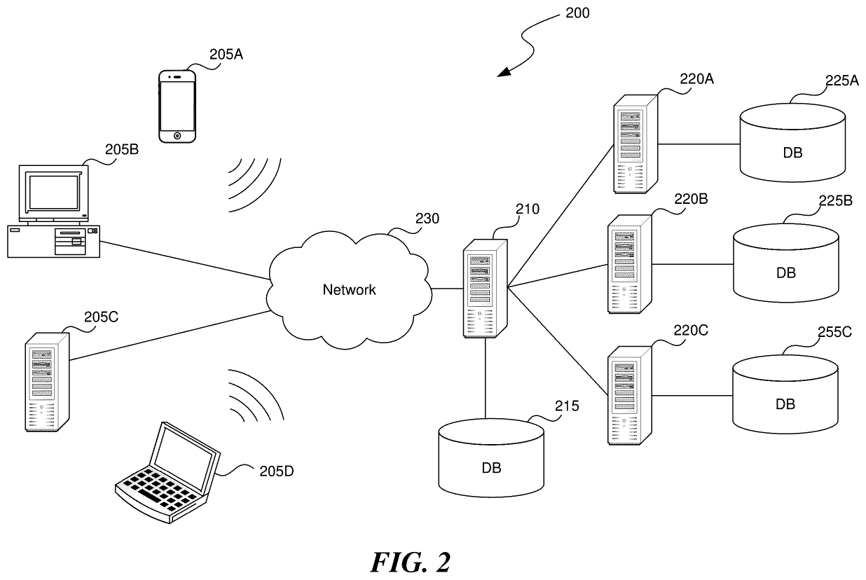

FIG. 2 is a block diagram illustrating an overview of an environment 200 in which some implementations of the disclosed technology can operate. Environment 200 can include one or more client computing devices 205A-D, examples of which can include device 100. Client computing devices 205 can operate in a networked environment using logical connections through network 230 to one or more remote computers, such as a server computing device.

In some implementations, server 210 can be an edge server which receives client requests and coordinates fulfillment of those requests through other servers, such as servers 220A-C. Server computing devices 210 and 220 can comprise computing systems, such as device 100. Though each server computing device 210 and 220 is displayed logically as a single server, server computing devices can each be a distributed computing environment encompassing multiple computing devices located at the same or at geographically disparate physical locations. In some implementations, each server 220 corresponds to a group of servers.

Client computing devices 205 and server computing devices 210 and 220 can each act as a server or client to other server/client devices. Server 210 can connect to a database 215. Servers 220A-C can each connect to a corresponding database 225A-C. As discussed above, each server 220 can correspond to a group of servers, and each of these servers can share a database or can have their own database. Databases 215 and 225 can warehouse (e.g., store) information such as images, video, audio, or other suitable application call data, parameters and architecture for machine learning model(s), tensor representations of data, and other suitable data. Though databases 215 and 225 are displayed logically as single units, databases 215 and 225 can each be a distributed computing environment encompassing multiple computing devices, can be located within their corresponding server, or can be located at the same or at geographically disparate physical locations.

Network 230 can be a local area network (LAN) or a wide area network (WAN), but can also be other wired or wireless networks. Network 230 may be the Internet or some other public or private network. Client computing devices 205 can be connected to network 230 through a network interface, such as by wired or wireless communication. While the connections between server 210 and servers 220 are shown as separate connections, these connections can be any kind of local, wide area, wired, or wireless network, including network 230 or a separate public or private network.

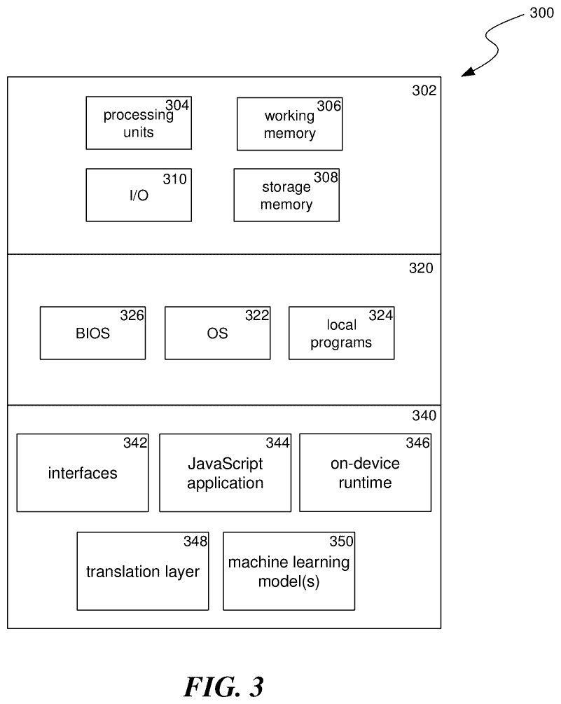

FIG. 3 is a block diagram illustrating components 300 which, in some implementations, can be used in a system employing the disclosed technology. The components 300 include hardware 302, general software 320, and specialized components 340. As discussed above, a system implementing the disclosed technology can use various hardware including processing units 304 (e.g. CPUs, GPUs, APUs, etc.), working memory 306, storage memory 308 (local storage or as an interface to remote storage, such as storage 215 or 225), and input and output devices 310. In various implementations, storage memory 308 can be one or more of: local devices, interfaces to remote storage devices, or combinations thereof. For example, storage memory 308 can be a set of one or more hard drives (e.g. a redundant array of independent disks (RAID)) accessible through a system bus or can be a cloud storage provider or other network storage accessible via one or more communications networks (e.g. a network accessible storage (NAS) device, such as storage 215 or storage provided through another server 220). Components 300 can be implemented in a client computing device such as client computing devices 205 or on a server computing device, such as server computing device 210 or 220.

General software 320 can include various applications including an operating system 322, local programs 324, and a basic input output system (BIOS) 326. Specialized components 340 can be subcomponents of a general software application 320, such as local programs 324. Specialized components 340 can include JavaScript application 344, on-device runtime 346, translation layer 348, machine learning model(s) 350, and components which can be used for providing user interfaces, transferring data, and controlling the specialized components, such as interfaces 342. In some implementations, components 300 can be in a computing system that is distributed across multiple computing devices or can be an interface to a server-based application executing one or more of specialized components 340. Although depicted as separate components, specialized components 340 may be logical or other nonphysical differentiations of functions and/or may be submodules or code-blocks of one or more applications.

JavaScript application 344 can be a JSI-compatible JavaScript runtime hosting an application that executes on a computing device configured for on-device machine learning execution. For example, JavaScript application 344 can be a React Native application, or any other JSI-compatible JavaScript application. Implementations of JavaScript application 344 pass software application calls to on-device runtime 346 via translation layer 348, such as software application calls that include data for machine learning model execution.

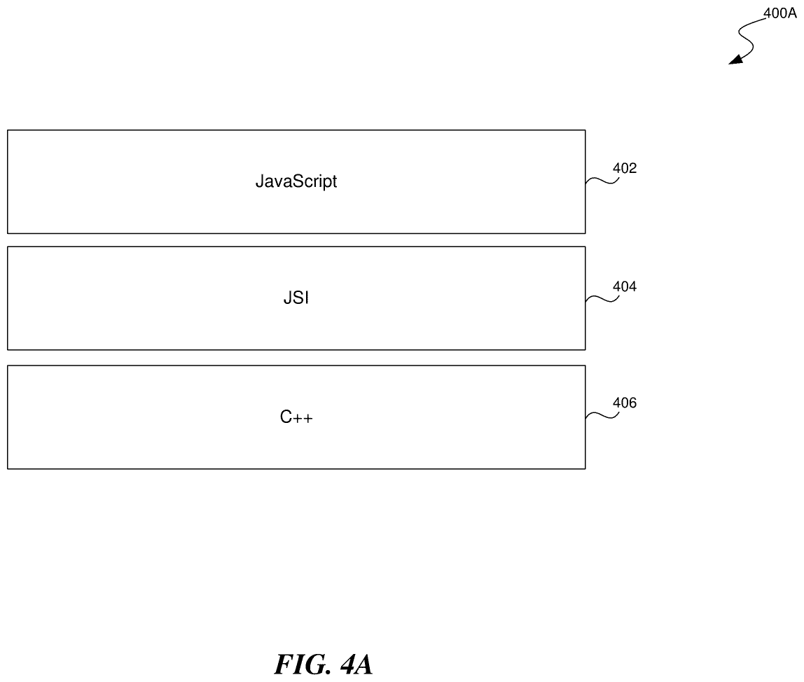

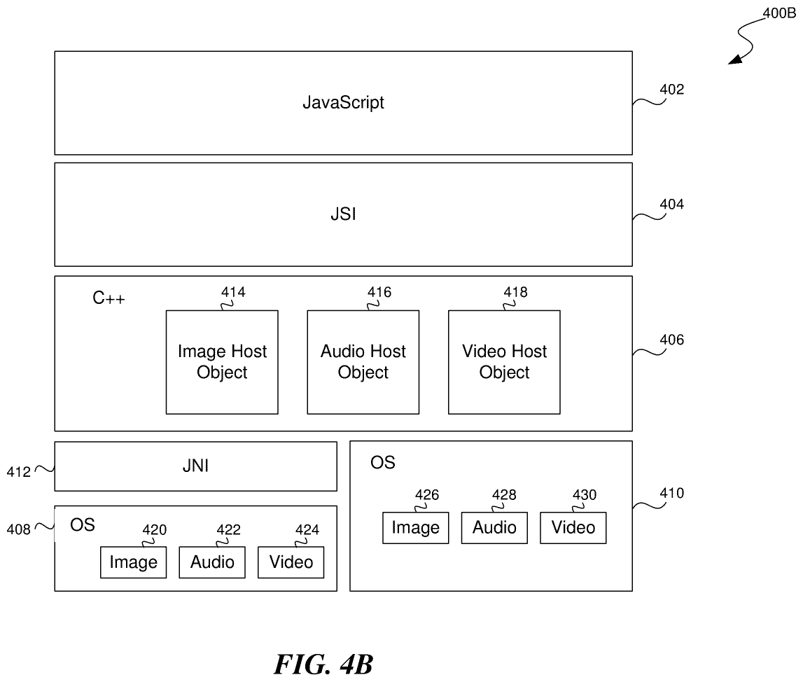

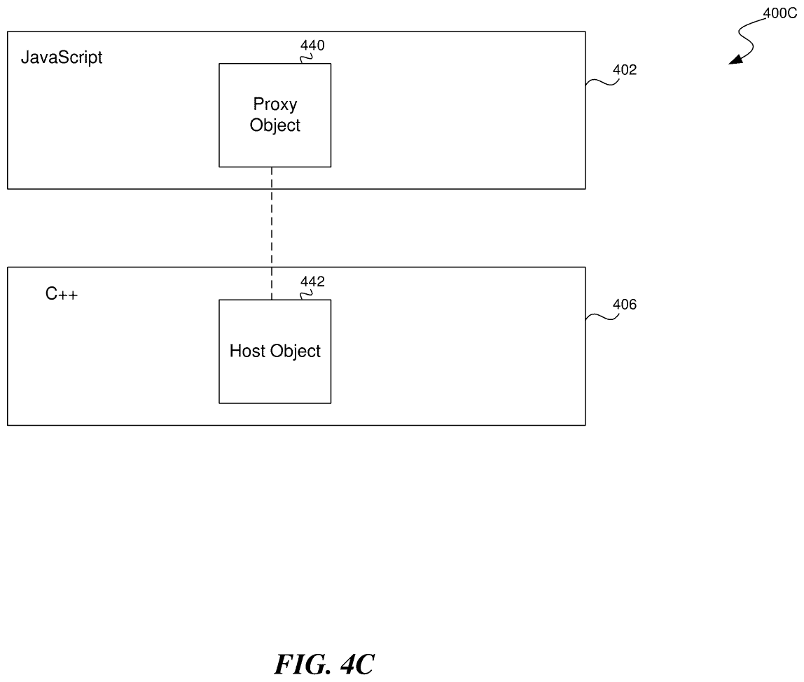

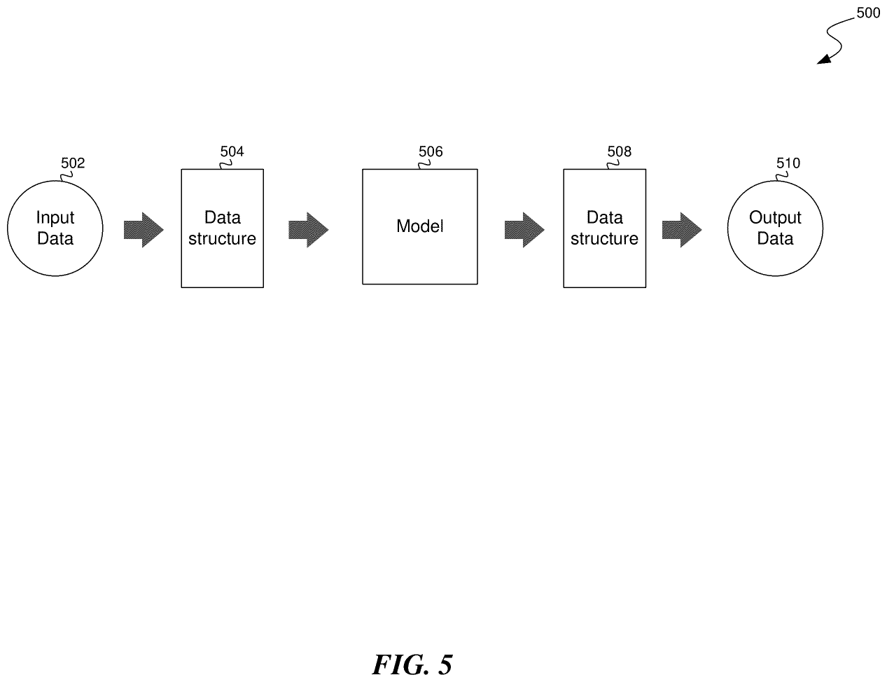

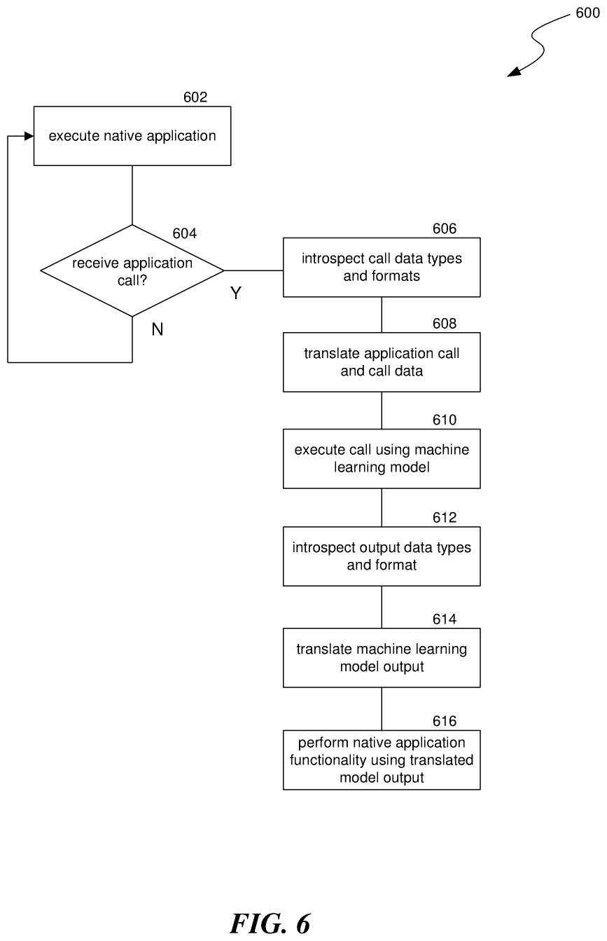

In some implementations, JavaScript application 344 can be compatible with a variety of data types, such as JavaScript data types (e.g., number, strings, arrays, etc.) and high-level data types, such as images, video, audio, and the like. In addition, the underlying file that some data types reference (e.g., image, video, audio, etc.) can be stored in a variety of formats, such as different image formats, video formats, audio formats, text formats, and the like. However, JavaScript data types and/or conventional file formats may not be compatible with on-device runtime 346 and/or machine learning model(s) 350. Translation layer 348 can translate JavaScript application 344 software calls to generate runtime compatible application calls and/or call data (e.g., tensor representations of call data). Implementations of on-device runtime 346 and machine learning model(s) 350 receive the runtime compatible calls and/or runtime compatible data, and perform model execution (e.g., machine learning inference) using the data. Implementations of translation layer 348 can then translate output data from machine learning model(s) 350 to JavaScript compatible data types and/or file formats and provide such output to JavaScript application 344. Additional details on JavaScript application 344 are provided below in relation to FIGS. 4A, 4B, 4C, 5 and process 600 of FIG. 6.

On-device runtime 346 can be any suitable runtime that can execute machine learning model(s) 350. In some implementations, on-device runtime 346 is a Java, Python, Basic, C, C #, C++ (e.g., PyTorch Mobile) runtime environment, or any other suitable on-device runtime for mobile devices. On-device runtime 346 can include software components that load, manage, and execute machine learning model(s) 350. For example, on-device runtime 346 can include application programming interfaces, libraries, and other suitable software structures that support machine learning execution (e.g., mathematical operations, data pipelining, tensor operations, machine learning inference operations, etc.). Additional details on on-device runtime 346 are provided below in relation to FIGS. 4A, 4B, 4C, 5 and process 600 of FIG. 6.

Translation layer 348 can translate data passed between JavaScript application 344 and on-device runtime 346. For data passed from JavaScript application 344 to on-device runtime 346 (e.g., input data for machine learning model(s) 350), translation layer 348 can translate and/or wrap the application call data from a JavaScript data types, high-level data type (e.g., images, video, audio, and other suitable data types), and/or conventional file formats to runtime compatible data types/file formats (e.g., C++ typed data types), machine learning model compatible data types/file formats (e.g., tensor representations), or any combination thereof. The machine learning model compatible data type(s) and/or file formats can be provided, as input, to machine learning model(s) 350 for execution (e.g., machine learning inference). Machine learning model(s) 350 can generate output data (e.g., tracked objects, augmented images, video, and/or audio, prediction data, or any other suitable machine learning model output), for example by applying a plurality of learned machine learning parameters to the input data.

For data passed from on-device runtime 346 to JavaScript application 344 (e.g., output data from machine learning model(s) 350), translation layer 348 can translate and/or wrap output data from the machine learning model compatible data type/file format to a JavaScript data type, high-level data type compatible with JavaScript application 344, file format compatible with JavaScript application 344 functionality, or any combination thereof. Implementations of translation layer 348 can support on-device machine learning model execution for JavaScript application 344 by performing multi-directional data translation that provides each processing module (e.g., JavaScript application 344 and Machine learning model(s) 350) compatible data. Additional details on translation layer 348 are provided below in relation to FIGS. 4A, 4B, 4C, 5 and process 600 of FIG. 6.

Machine learning model(s) 350 can be any suitable machine learning model configured for execution by on-device runtime 346. For example, on-device runtime 346 and machine learning model(s) 350 can be loaded onto an edge device, smart home device, mobile device (e.g., smartphone), or any other suitable device that does not reside in the cloud. Examples of machine learning model(s) 350 include: neural networks (e.g., convolutional neural networks, recurrent neural networks, transformer networks, etc.), generative adversarial networks (GANs), encoder/decoder networks, support vector machines, decision trees, Parzen windows, Bayes networks, clustering models, reinforcement learning models, probability distributions, decision tree forests, and others. Models can be configured for various situations, data types, sources, and output formats.

In some implementations, machine learning model(s) 350 may undergo one or more optimization techniques to optimize a conventional machine learning model for mobile execution. Example optimization techniques can include fusing (e.g., reducing model size by fusing layers), quantization (e.g., reducing model size by approximation), and other suitable optimization techniques. Machine learning model(s) 350 can perform any suitable machine learning functionality for JavaScript application 344 (e.g., via translation layer 348 and On-device runtime 346), such as media (e.g., image, video, audio, etc.) augmentation, object tracking, data classification (e.g., image or object classification), natural language processing, machine prediction, any other suitable machine learning functionality, or any combination thereof.

In some implementations, the machine learning model(s) 350 loaded on-device can be pretrained. For example, supervised learning, semi-supervised learning, unsupervised learning, reinforcement learning, or any other suitable learning can train parameters (e.g., weights) of machine learning model(s) 350. One or more pretrained machine learning model(s) 350 can be loaded on-device to perform inference execution. For example, one or more of machine learning model(s) 350 can be pretrained to track objects in video frames, and the inference execution using this pretrained machine learning model can include: inputting video frames (e.g., tensor representations) to the pretrained model; and receiving output video frames with bounding boxes and/or other suitable output data that locates objects within the video frames.

An example of machine learning model(s) 350 that is a pretrained on-device model is a GAN. GANs can include two neural networks, a generator and a discriminator. In training, a GAN's generator is trained to fool the GAN's discriminator. A GAN can be trained using a variety of techniques, including unsupervised learning, semi-supervised learning, fully supervised learning, and/or reinforcement learning. In some implementations, an on-device GAN can be trained to generate augments for visual frames (e.g., images or video). Example augments can include visual effects (e.g., overlays, virtual images/objects, color and/or visual style effects, such as filters, etc.), object tracking and labeling, and other suitable augments.

Implementations perform on-device machine learning execution (e.g., inference) with a GAN (or any other suitable machine learning model(s) 350) at a mobile device using one or more mobile designed processors (e.g., multi-core central processing unit (CPU), CPU designed for smartphones, etc.). For example, the mobile designed processor(s) may not include graphics processing units (GPUs). In some implementations, one or more machine learning model(s) 350 can be trained on-device, and/or transfer learning can be performed on-device. Additional details machine learning model(s) 350 are provided below in relation to FIGS. 4A, 4B, 4C, 5 and process 600 of FIG. 6.

Implementations support on-device machine learning execution using a translation layer that translates JavaScript software application call data to achieve interoperability with on-device machine learning models. The on-device machine learning execution refers to machine learning performed on a mobile device, client device, edge device, or any other suitable device that is not located in the cloud. Conventional machine learning is often performed at a server or other suitable cloud device that includes sophisticated software environments and/or large quantities of available computing resources. Machine learning in the context of constrained computing resources and less sophisticated software environments presents a unique set challenges.

Implementations leverage on-device runtime environments, efficient machine learning models, and a translation layer to provide machine learning functionality to JavaScript applications running natively on-device. Software applications running natively on a device (e.g., smartphones, Internet of Things (IoT) devices, smart home devices, and the like) often interact with data formatted in a manner that is conducive to the software application. For example, a React Native JavaScript application can comprise data types for audio, video, images, and text that support interactions with the application, such as execution of the audio, execution of the video, and/or display of the image. Some object data types also refer to underlying data files, such as image files, video files, audio files, text documents, and the like. These data files are stored in file formats that are conducive to JavaScript application functionality, such as displaying an image, playing a video, playing audio, and the like. However, these data types and/or file formats are often incompatible with machine learning execution. For example, machine learning models may interact with the same type of data in a tensor representation.

Data files, such as images, video, audio, text, and other data, are often stored in a file format that does not comprise a tensor representation of the data, for example to support efficient interactions with JavaScript software applications. Implementations of a translation layer can convert these data file into tensor representations that are compatible with machine learning model execution. In some implementations, the translation layer can translate any suitable data type (e.g., media data types, text data type, etc.) or data format compatible with a JSI-compatible software application into a data type or data format that complies with the compatibility of a loaded machine learning model.

Output from machine learning model(s) is also often in a format that is compatible with the machine learning model(s), such as a tensor representation, but incompatible with a native JavaScript software application. For example, software application functionality, such as displaying an image/video file or playing an audio file, is often inefficient or otherwise impractical using tensor representations of data. Implementations of the translation layer can also convert tensor representations of data into conventional data types (e.g., JavaScript image, audio, video, or text data types) and/or data formats that are compatible with software application functionality. According, the translation layer can support machine learning model functionality alongside JavaScript application functionality by providing each component compatible data.

FIGS. 4A, 4B and 4C are conceptual diagrams of software architecture that translates application calls for on-device machine learning execution. Diagram 400A includes JavaScript component 402, JavaScript Interface (JSI) 404, and C++ component 406. JavaScript component 402 can comprise a native JavaScript application that is run on-device, such as a React Native application.

Implementations of JavaScript component 402 includes JavaScript code and portions of React Native, a cross-platform JavaScript library for building user interfaces a cross-platform JavaScript library for building user interfaces. For example, JavaScript component 402 can include a JavaScript runtime environment that executes JavaScript code (e.g., a JavaScript React Native application) on-device. JSI 404 is an interface layer between JavaScript component 402 and a component written in a code other than JavaScript, such as C++ component 406. For example, JSI 404 can support method and/or object creation and registration with a JavaScript runtime environment (part of JavaScript component 402) for methods and/or objects that are part of other programming languages, such as C++, Java, and the like. JSI 404 can be a component of a React Native framework loaded on-device.

In some implementations, C++ component 406 can include a C++ on-device runtime environment, such as PyTorch Mobile. Using JSI 404, JavaScript code can reference C++ host objects and invoke C++ methods using the host objects. For example, JavaScript code (JavaScript component 402) can access PyTorch Mobile methods (at C++ component 406) using referenced C++ host objects. Diagram 400B includes JavaScript component 402, JavaScript Interface (JSI) 404, C++ component 406, first operating system 408, second operating system 410, Java Native Interface (JNI) 412, image host object 414, audio host object 416, and video host object 418.

C++ component 406 can include image host object 414, audio host object 416, and video host object 418. For example, JavaScript code executed at JavaScript component 402 can, via JSI 404, reference image host object 414, audio host object 416, and/or video host object 418, and call one or more C++ library functions (e.g., PyTorch mobile library functions) using the host object reference. For example, one or more of the C++ library functions and C++ host objects can be registered with a JavaScript runtime (part of JavaScript component 402) such that the JavaScript code can make a JavaScript call that corresponds to the one or more C++ library functions using a JavaScript object that references a C++ host object.

Diagram 4000 includes JavaScript component 402, C++ component 406, proxy object 440, and host object 442. Proxy object 440 can be a JavaScript object that references host object 442, or the C++ host object. For example, host object 442 can be registered with a JavaScript runtime environment (part of JavaScript component 402) which can expose host object 442 to JavaScript code as proxy object 440. The JavaScript code can call one or more methods or functions that are part of C++ component 406 using proxy object 440. The connection between proxy object 440 and host object 442 is leveraged to support executing C++ method or function calls on behalf of the JavaScript code/proxy object 440 using host object 442.

For example, a C++ runtime environment that is part of C++ component 406 (e.g., PyTorch Mobile) can implement the C++ library function that corresponds to the JavaScript call using host object 442. In some implementations, the C++ library function can be a PyTorch Mobile machine learning model function, such as a machine learning model compiler and/or initializer (e.g., torch.jit._load_for_mobileo), a machine learning model inference function (e.g., model_name.forward(input_data)), a machine learning model mathematical operation (e.g., output.argmaxo), or any other suitable function. Output from the machine learning model function can be returned to the JavaScript code that first made the JavaScript call via proxy object 440.

Referring back to diagram 400B, Java Native Interface (JNI) 412 is a foreign function interface that supports Java code (e.g., executed by the Java Virtual machine (JVM)) interoperability with applications/software that is non-Java code. For example, JNI 412 can support application calls and library calls between Java code and non-Java code (e.g., C, C++, etc.). In some implementations, JNI 412 can support interoperability between first operating system 408, such as a Java mobile operating system (e.g., Android), and C++ component 406, which can include a C++ on-device runtime environment, such as PyTorch Mobile, and the like. Implementations of second operating system 410 can be a mobile operating system that is written in a programming language other than Java (e.g., C, C++, Objective C, Swift, etc.). For example, second operating system 410 can be an iOS operating system, or any other suitable mobile operating system.

Implementations of a translation layer can convert data types and/or data files that are part of a JavaScript native application software call via proxy object 440. For example, one or more method/library calls can be registered with the JavaScript runtime environment associated with proxy object 440. These method/library calls can correspond to C++ library functions and/or methods that correspond to host object 442. In some implementations, the registered method/library calls include call data, such as JavaScript data type data, data file(s), or other suitable call data. Implementations of the translation layer can translate: JavaScript data types for compatibility with a C++ runtime environment and/or on-device machine learning models; and data file formats for compatibility with the C++ runtime environment and/or on-device machine learning models. Implementations of the translation layer can also translate output from the machine learning model(s) back to a JavaScript compatible data type(s) and/or file format(s) when returning data to the JavaScript code in response to the JavaScript native application software call. The translation layer can be part of JavaScript component 402, JSI 404, C++ component 406, or any combination thereof.

In some implementations, host object 442 can be any type of C++ host object that comprises method and/or library function calls. Host object 442 can be part of a C++ framework, such as PyTorch Mobile. Examples of host object 442 include tensor host object, blob host object, image host object, audio host object, text host object, video host object, one or more framework specific host objects (e.g., torch host object, JIT host object, Torchvision host object, IValue host object, and other suitable PyTorch Mobile host objects). Similarly, examples of proxy object 440 can include tensor proxy object, blob proxy object, image proxy object, audio proxy object, text proxy object, video proxy object, one or more framework specific proxy objects (e.g., torch proxy object, JIT proxy object, Torchvision proxy object, IValue proxy object, and other suitable PyTorch Mobile proxy objects).

For example, the torch proxy object and torch host object can expose, to the JavaScript native application, a set of tensor operations, such as creating and calculating tensors. Pseudocode examples of the methods/functions include tensor(Data), add(tensorRef, tensorRef), multiply(tensorRef, tensorRef), rand(size), ones(size), reshape(tensorRef, size), size(tensorRef), among others. In another example, the tensor proxy object and tensor host object can provide a tensor object for the JavaScript native application. In another example, the blob proxy object and blob host object can provide a blob object for the JavaScript native application that is a pointer to a memory location (e.g., media blob).

In another example, a torchvision host object and torchvision proxy object can expose, to the JavaScript native application, functions from a torchvision module. Pseudocode examples of the methods/functions include Compositions: Compose( ); Transforms on Image and Torch (e.g., tensor): ImageToTensor( ) (e.g., transforms.PILToTensor), ConvertImageDtype( ), Normalize( ), ColorJitter( ), FiveCrop( ), Grayscale( ), Pad( ), RandomRotation( ), Scale( ), GaussianBlur( ), RandomInvert( ); Transforms on Images: RandomChoice( ), ImageToTensor( ), ConvertImageDtype( ); Transforms on Tensor: LinearTransformation, Normalize(mean, std, inplace=False), RandomErasing(p=0.5, scale=(0.02, 0.33), ratio=(0.3, 3.3), value=0, inplace=False); Conversion Transforms: ToImage( ), ToTensor( ); Generic Transforms; Automatic Augmentation Transforms; Functional Transforms; among others.

The image proxy/host object, video proxy/host object, and audio proxy/host object can implement a MediaProxy Object. For example, the media proxy object can implement the following pseudocode examples of functions and properties: data( ), arrayBuffer( ), size( ), type, toString( ), among others. The image proxy/host object can also expose, to the JavaScript native application, the following example pseudocode functions (in addition to the MediaProxy Object functions): getHeight( )*pixel, getWidth( )*pixel. The video proxy/host object can also expose, to the JavaScript native application, the following example pseudocode functions (in addition to the MediaProxy Object functions): getHeight( )*pixel, getWidth( )*pixel, getDuration( ). The audio proxy/host object can also expose, to the JavaScript native application, the following pseudocode example function (in addition to the MedixProxy Object functions): getDuration( ).

In another example, the text proxy/host object can provide an object for text (e.g., text input) and expose, to the JavaScript native application, the following example functions: getLength( ). In another example, the tensor proxy/host object can provide a tensor object and expose, to the JavaScript native application, the following example functions: select(int[ ]), crop( ), resize( ), getSize( ).

In some implementations, JavaScript native software application calls that are implemented via a proxy object linked to a host object can be machine learning model library functions that include call data, such as images, videos, audio, text, JavaScript data typed data, and other suitable data. Implementations of a translation layer can translate (e.g., convert, wrap, etc.) call data to provide data compatible with one or more on-device machine learning models.

Implementations achieve JavaScript Application call translation that is agnostic to the mobile platform as long as the platform is compatible with JavaScript Interface (JSI). For example, integrating on-device machine learning for multiple operating systems (e.g., Android and iOS) can be achieved by writing a single integration in JavaScript. Conventional options require writing code for the on-device integration layer for each platform individually.

FIG. 5 is a conceptual diagram of data translations between a native application and machine learning model(s). Diagram 500 includes input data 502, input data structure representation 504, machine learning model 506, output data structure representation 508, and output data 510. Implementations of a translation layer can translate input data 502 into input data structure representation 504, or a representation compatible with machine learning model 506. Input data 502 can be part of a JavaScript application call and include an image, video, audio, another suitable JavaScript data type, or any other suitable data compatible with a native software application (e.g., JavaScript React Native application). The translation layer can translate input data 502 into input data structure representation 504 by wrapping the data in a wrapper object and/or converting the data from its initial representation (e.g., JavaScript data type, conventional data file, etc.) to the machine learning compatible representation (e.g., data structure representation). In some implementations, a data structure representation of data can be a tensor representation, an array or list representation, a dictionary or map representation, a tuple representation, or any combination thereof.

In some implementations, machine learning model 506 can be preconfigured (e.g., trained) to generate formatted output data using formatted input data. For example, machine learning model 506 can be configured to take, as input, media data (e.g., image, plurality of images, such as video, etc.) in a predefined format that meets a formatting criteria (e.g., a data structure/tensor representation that comprises predefined dimensions/sizing). Similarly, machine learning model 506 can be configured to generate, using the media data formatted according to the predefined format, output data (e.g., augmented media data, etc.) in a predefined format (e.g., data structure/tensor representation that comprises predefined dimensions/sizing).

In some implementations, the translation layer can translate input data 502 into input data structure representation 504 to comply with the data format for machine learning model 506. In an example, input data 502 can include one or more images (e.g., static image, video frames, etc.) and the predefined data format can include dimensions and sizing for a data structure representation of the one or more images. The translation layer can perform one or more transformations on input data 502 to generate input data structure representation 504, such as media.tensorFromImage( ) or image.toTensor( ), or other suitable transformations. Similarly, input data 502 can include one or more audio files, text files, and the like, and the predefined data format can include dimensions and sizing for a data structure representation of the data. The translation layer can perform one or more transformations on input data 502 to generate input data structure representation 504, such as ToTensor( ), or other suitable transformations.

In some implementations, the translation layer may perform data introspection on input data 502 to introspect data types and/or data files. For example, input data 502 can include a pointer to a data blob, such as an unidentified data file in memory. The translation layer can perform introspection on input data 502 to support translation to on-device runtime environment compatible data representations, such as C++ data types and/or data structure representations. Implementations can introspect the data types or data files of input data 502 using one or more introspection methods/functions that test a variety of data types/files (e.g., functions to test whether data is an image, audio, video, text file, etc.). For example, one of the method calls can return a positive value that indicates the data type(s) and/or type(s) of data file(s) (e.g., image, audio, video, text file, etc.) for input data 502. The translation layer can then perform the relevant transformations for the introspected data types and/or data files to translate input data 502 into input data structure representation 504.

Machine learning model 506 can process input data structure representation 504 to generate output data structure representation 508. For example, machine learning model 506 can include a plurality of machine learning model parameters (e.g., weights) configured during a training phase. Machine learning model 506 can generate output data structure representation 508 by applying the machine learning model parameters to input data structure representation 504. In some implementations, the machine learning model parameters comprise weights of one or more trained neural network (e.g., convolutional neural network, transformer network, generative adversarial network (GAN), etc.), and applying the parameters includes performing mathematical operations on input data structure representation 504 using the weights as the data progresses through the trained neural network.

Output data structure representation 508 can be any suitable data structure representation formatted according to the design/architecture of machine learning model 506. Implementations of the translation layer can translate output data structure representation 508 to output data 510, for example to return output data 510 to the calling JavaScript application in a compatible format (e.g., compatible image file, video file, audio file, text file, etc.). Output data 510 can include one or more images (e.g., static image, video frames, etc.), audio, text, and the like. The translation layer can perform one or more transformations on output data structure representation 508 to generate output data 510, such as media.tensorToImage( ), media.tensorToAudio( ), media.tensorToVideo( ), or other suitable transformations.

In some implementations, the translation layer may perform data introspection on output data structure representation 508 to introspect the data represented by the data structure representation. For example, output data structure representation 508 can represent an image, video, audio, text, or any other suitable data. The translation layer can perform introspection on output data structure representation 508 to support translation to JavaScript application compatible data types and/or data file formats. Implementations can introspect the data represented by output data structure representation 508 using one or more introspection methods/functions that test a variety of data types/files (e.g., functions to test whether the data structure representation is an image, audio, video, text file, etc.). For example, one of the method calls can return a positive value that indicates the data represented by output data structure representation 508. The translation layer can then perform the relevant transformations for the introspected data types and/or data files to translate output data structure representation 508 into output data 510. The calling JavaScript application can perform software functions with output data 510 that is returned in response to the software call. These functions can include displaying an image or video, playing an audio file, processing and/or display a text file, or any other suitable software functions.

In some implementations, the translation layer can translate between JavaScript compatible data types and C++ compatible data types (e.g., to JavaScript from C++, from C++ to JavaScript). Table 1 illustrates a variety of example translations that can be performed by the translation layer. For example, JavaScript application call data can be translated to one or more C++ compatible data types, a C++ compatible data type output by machine learning model(s) can be converted to one or more JavaScript application compatible data types, and the like.

TABLE 1

| JavaScript | C++ |
|---|---|
| null/undefined | None |
| number | int |
| number | double |
| string | string |
| | stringRef |
| boolean | boolean |
| Array (or unpack to multiple return values) | Tuple |
| Array | GenericList |
| | GenericListRef |
| Map | GenericDict |
| | GenericDictRef |
| | Blob |
| TensorHostObject | Tensor |
| | Object |
| Array | IntList |
| | IntListRef |
| Array | DoubleList |
| | DoubleListRef |
| Array | BoolList |
| | BoolListRef |
| Array | TensorList |
| | TensorListRef |
| | Future |
| | Device |
| Number | Scalar |

A “machine learning model,” as used herein, refers to a construct that is trained using training data to make predictions or provide probabilities for new data items, whether or not the new data items were included in the training data. For example, training data for supervised learning can include items with various parameters and an assigned classification. A new data item can have parameters that a model can use to assign a classification to the new data item. As another example, a model can be a probability distribution resulting from the analysis of training data, such as a likelihood of a service being used a given a location or set of users based on an analysis of previous user service selections.

Some machine learning models can be trained with supervised learning. For example, a convolutional neural network can be trained to detect and track object in streaming video. The training data can include visual frames as input and a desired output, such as object boundary labels (e.g., bounding boxes). An instance of training data, such as a visual frame, can be provided to the model. Output from the model can be compared to the desired output for that visual frame and, based on the comparison, the model can be modified, such as by changing weights between nodes of the neural network or parameters of the functions used at each node in the neural network (e.g., applying a loss function). After applying the visual frames and labels in the training data and modifying the model in this manner, the model can be trained to track objects.

Those skilled in the art will appreciate that the components illustrated in FIGS. 1-3, 4A, 4B, 4C, and 5 described above, and in each of the flow diagrams discussed below, may be altered in a variety of ways. For example, the order of the logic may be rearranged, substeps may be performed in parallel, illustrated logic may be omitted, other logic may be included, etc. In some implementations, one or more of the components described above can execute one or more of the processes described below.

FIG. 6 is a flow diagram illustrating a process used in some implementations for translating application calls for on-device machine learning execution. In some implementations, process 600 can be performed in response to an application call from a native application (e.g., JavaScript application). In some implementations, process 600 can be performed at a computing device that supports on-device machine learning execution.

At block 602, process 600 can execute a native application. For example, the native application can be a React Native JavaScript application executing on-device. The device can be a mobile device (e.g., smartphone), edge device, smart home device, IoT device, wearable device, or any other suitable device that is not located in the cloud. The JavaScript application can be executed by a JSI-compatible JavaScript runtime environment, such as a JavaScript runtime environment that is part of the React Native framework.

At block 604, process 606 detect an application call for execution using one or more machine learning models. For example, the React Native JavaScript application can make a software application call to one or more machine learning models. The software application call can comprise call data (e.g., image, video, audio, text, or a combination thereof) for the machine learning model(s). For example, a camera of a mobile device can capture on or more images/video frames, and the JavaScript application can include the captured images/video frames in the software application call. In some implementations, the software application call can comprise one or more library calls that perform functions using the machine learning model(s).

In some implementations, the machine learning model(s) can execute in an on-device runtime environment that is not compatible with the React Native JavaScript application. For example, the on-device runtime environment can be a C++ runtime, such as a runtime environment from the PyTorch Mobile Framework. Within the on-device runtime environment, machine learning model library methods and functions can be called and executed.

In some implementations, the software application call can be accomplished via a proxy object registered with the JavaScript framework (e.g., JavaScript runtime environment). For example, the proxy object can be connected to a host object that can call functions and methods within the on-device runtime environment (e.g., C++ runtime environment). In this example, the React Native JavaScript application can make the software application call relative to the proxy object, and the corresponding host object can implement the software application call within the on-device runtime environment to accomplish machine learning model functionality.

When an application call for execution using machine learning model(s) is detected, process 600 can progress to block 606. When an application call for execution using machine learning model(s) is not detected, process 600 can loop back to block 602, where native application execution can continue.

At block 606, process 600 can introspect software application call data. For example, the software application call can comprise JavaScript data types and/or formatted data files, such as image, audio, video, and/or text data types or data files. These data types and/or data files can be introspected to support translation to on-device runtime environment compatible data representations, such as C++ data types and/or data structure representations (e.g., tensor representation, array or list representation, dictionary or map representation, tuple representation, or any combination thereof). Implementations can introspect the data types or data files of a JavaScript application call using one or more introspection methods that test a variety of data types/files (e.g., methods to test whether data is an image, audio, video, text file, etc.). For example, one of the method calls can return a positive value that indicates the data type and/or type of data file (e.g., image, audio, video, text file, etc.).

At block 608, process 600 can translate the application call for execution using machine learning model(s). For example, the software application call data can be translated to translated call data that is compatible with the machine learning model(s). In some implementations, the translated call data comprises a data structure representation of the call data. Translating the call data can include executing one or more library functions that translate a data type or format (e.g., image, audio, video, text file, etc.) to a data structure representation of the data type or format.

Implementations introspect data types and/or data formats for software call data, and the translation can include translating the call data according to the introspected data type or format. For example, call data can include a data blob or unidentified data, the introspected data type can determine the type or format for the unidentified data (e.g., image, audio, video, text file, etc.), and one or more transformations can transform the data from its initial form (e.g., data file formatted for a JavaScript application) to a data structure representation using transformations relevant to the introspected type/format.

In some implementations, the software application call can include a translation specification for the call data, include data type/format, library functions, sizing, other suitable transformation information, or any combination thereof. In this example, the call data can be translated according to the translation specification to generate the data structure representation.

At block 610, process 600 can execute the translated application call using the machine learning model(s). For example, executing the translated application call can include executing library functions using machine learning model(s) and the translated call data (e.g., data structure representation of data). The translated call data can be input to the machine learning model(s) and the machine learning model(s) can apply learned parameters (e.g., weights) to the input data to generate output data. In some implementations, the machine learning model functionality can include object tracking in image(s), object classification in image(s), generating augmented visual effects in image(s), natural language processing, audio processing, or any other suitable machine learning functionality. In some implementations, the data output by the machine learning model(s) can include text, audio, video, images, and other suitable data in a data structure representation.

In some implementations, the translated application call is performed via a host object that operates in an on-device runtime environment (e.g., C++ runtime environment). For example, the JavaScript application can make the initial software application call relative to a proxy object connected to the host object. The initial JavaScript call can be translated to a C++ library/method software call relative to the host object, and the translated software application call can perform the machine learning model execution within the on-device runtime environment.

At block 612, process 600 can introspect machine learning model output data types. For example, data output by the machine learning model(s) can represent an image, video, audio, text, or any other suitable data. The machine learning model output data can comprise machine learning model compatible data types and data formatting, such as C++ data types, data structure representations, and the like. These types/formats can be introspected to support translation to JavaScript compatible data types/formats. Implementations can introspect the data represented by the output data using one or more introspection methods/functions that test a variety of data types/formats (e.g., functions to test whether the data structure representation is an image, audio, video, text, tensor, etc.). For example, one of the method calls can return a positive value that indicates the data represented by the output data. In some implementations, an on-device runtime environment and framework (e.g., PyTorch Mobile) can support introspection methods for machine learning model output data.

At block 614, process 600 can translate the output data from the machine learning model(s). For example, the output data (e.g., data structure representation) can be translated to translated output data that is compatible with the JavaScript application (e.g., JavaScript compatible type or data file, such as image, audio, video, text, and the like). Translating the output data can include executing one or more library functions that translate a data structure representation to a data type or format (e.g., image, audio, video, text file, etc.).

Implementations introspect data types and/or data formats for output data, and the translation can include translating the output data according to the introspected data type or format. For example, output data can include a data structure representation of data, the introspected data type can determine the type or format for the data structure representation of data (e.g., image, audio, video, text file, etc.), and one or more transformations can transform the data structure representation to a JavaScript application compatible data type or format using transformations relevant to the introspected type/format.

At block 616, process 600 can perform native application functionality using the translated machine learning model output. For example, the translated machine learning model output can include an image that is displayed by the JavaScript application, a video that is played by the JavaScript application, text that is processed or displayed by the JavaScript application, audio that is played by the JavaScript application, any other suitable application functionality using data output by machine learning model(s), or any combination thereof.

Several implementations of the disclosed technology are described above in reference to the figures. The computing devices on which the described technology may be implemented can include one or more central processing units, memory, input devices (e.g., keyboard and pointing devices), output devices (e.g., display devices), storage devices (e.g., disk drives), and network devices (e.g., network interfaces). The memory and storage devices are computer-readable storage media that can store instructions that implement at least portions of the described technology. In addition, the data structures and message structures can be stored or transmitted via a data transmission medium, such as a signal on a communications link. Various communications links can be used, such as the Internet, a local area network, a wide area network, or a point-to-point dial-up connection. Thus, computer-readable media can comprise computer-readable storage media (e.g., “non-transitory” media) and computer-readable transmission media.

Reference in this specification to “implementations” (e.g. “some implementations,” “various implementations,” “one implementation,” “an implementation,” etc.) means that a particular feature, structure, or characteristic described in connection with the implementation is included in at least one implementation of the disclosure. The appearances of these phrases in various places in the specification are not necessarily all referring to the same implementation, nor are separate or alternative implementations mutually exclusive of other implementations. Moreover, various features are described which may be exhibited by some implementations and not by others. Similarly, various requirements are described which may be requirements for some implementations but not for other implementations.

As used herein, being above a threshold means that a value for an item under comparison is above a specified other value, that an item under comparison is among a certain specified number of items with the largest value, or that an item under comparison has a value within a specified top percentage value. As used herein, being below a threshold means that a value for an item under comparison is below a specified other value, that an item under comparison is among a certain specified number of items with the smallest value, or that an item under comparison has a value within a specified bottom percentage value. As used herein, being within a threshold means that a value for an item under comparison is between two specified other values, that an item under comparison is among a middle specified number of items, or that an item under comparison has a value within a middle specified percentage range. Relative terms, such as high or unimportant, when not otherwise defined, can be understood as assigning a value and determining how that value compares to an established threshold. For example, the phrase “selecting a fast connection” can be understood to mean selecting a connection that has a value assigned corresponding to its connection speed that is above a threshold.

As used herein, the word “or” refers to any possible permutation of a set of items. For example, the phrase “A, B, or C” refers to at least one of A, B, C, or any combination thereof, such as any of: A; B; C; A and B; A and C; B and C; A, B, and C; or multiple of any item such as A and A; B, B, and C; A, A, B, C, and C; etc.

Although the subject matter has been described in language specific to structural features and/or methodological acts, it is to be understood that the subject matter defined in the appended claims is not necessarily limited to the specific features or acts described above. Specific embodiments and implementations have been described herein for purposes of illustration, but various modifications can be made without deviating from the scope of the embodiments and implementations. The specific features and acts described above are disclosed as example forms of implementing the claims that follow. Accordingly, the embodiments and implementations are not limited except as by the appended claims.

Any patents, patent applications, and other references noted above are incorporated herein by reference. Aspects can be modified, if necessary, to employ the systems, functions, and concepts of the various references described above to provide yet further implementations. If statements or subject matter in a document incorporated by reference conflicts with statements or subject matter of this application, then this application shall control.

I/We claim:

1. A method for translating application calls for on-device machine learning execution, the method comprising:
receiving, from a native JavaScript application, a software call that includes software call data comprising one or more of an image, a video, audio, text, or a combination thereof;
translating, by an interface layer, the software call data to translated call data compatible with one or more on-device machine learning models, wherein the translated call data comprises a data structure representation of the software call data, and the data structure representation comprises a tensor representation, an array or list representation, a dictionary or map representation, a tuple representation, or any combination thereof; and
executing, via a non-JavaScript on-device runtime environment, the software call using the one or more on-device machine learning models, wherein the executing generates an output from the one or more on-device machine learning models using the data structure representation of the call data.

2. The method of claim 1, further comprising:
translating, by the interface layer, the output from the one or more on-device machine learning models to JavaScript compatible data, the JavaScript compatible data comprising one or more of an image, a video, audio, text, or a combination thereof; and
performing, by the native JavaScript application, one or more software functions using the JavaScript compatible data.

3. The method of claim 1, wherein the non-JavaScript on-device runtime environment comprises a C++ runtime environment.

4. The method of claim 3, wherein the software call comprises one or more JavaScript calls and corresponds to one or more C++ library functions, the C++ library functions are registered with an on-device JavaScript runtime environment that executes the native JavaScript application, and the one or more JavaScript calls are translated to one or more calls to the C++ library functions.

5. The method of claim 4, wherein executing the software call using the one or more on-device machine learning models comprises executing, via the non-JavaScript on-device runtime environment, the translated one or more C++ library function calls using the one or more on-device machine learning models and the data structure representation of the call data.

6. The method of claim 1, wherein the receiving, translating, and executing is performed on-device at a mobile computing device or an edge computing device.

7. The method of claim 1, wherein the software call data comprises an input image, audio, or video and the output from the one or more on-device machine learning models comprises an augmented version of the input image, audio, or video.

8. The method of claim 7, wherein the input image or video is translated, at the translation layer, to a data structure representation of the input image, audio, or video, and the on-device machine learning models generate the augmented version of the input image, audio, or video using the data structure representation of input image, audio, or video.

9. The method of claim 8, wherein the augmented version of the input image, audio, or video output by the on-device machine learning models comprises a data structure representation of the augmented version of the input image, audio, or video, the data structure representation of the augmented version of the input image, audio, or video is translated, by the interface layer, to a JavaScript compatible augmented version of the input image, audio, or video, and the native JavaScript application performs one or more software functions using the JavaScript compatible augmented version of the input image, audio, or video.

10. The method of claim 8, further comprising:
introspecting a data type for the software call data to determine the input image, audio, or video type; and
translating the input image, audio, or video type into the data structure representation of the input image, audio, or video according to the introspected data type.

11. The method of claim 1, further comprising:
introspecting a data type for the output from the one or more on-device machine learning models;
translating, by the interface layer, the output from the one or more on-device machine learning models to JavaScript compatible data according to the introspected data type, the JavaScript compatible data comprising one or more of an image, a video, audio, text, or a combination thereof; and
performing, by the native JavaScript application, one or more software functions using the JavaScript compatible data.

12. A computer-readable storage medium storing instructions that, when executed by a computing system, cause the computing system to perform a process for translating application calls for on-device machine learning execution, the process comprising:
receiving, from a native JavaScript application, a software call that includes software call data comprising one or more of an image, a video, audio, text, or a combination thereof;
translating, by an interface layer, the software call data to translated call data compatible with one or more on-device machine learning models, wherein the translated call data comprises a data structure representation of the software call data, and the data structure representation comprises a tensor representation, an array or list representation, a dictionary or map representation, a tuple representation, or any combination thereof; and
executing, via an on-device runtime environment, the software call using the one or more on-device machine learning models, wherein the executing generates an output from the one or more on-device machine learning models using the data structure representation of the call data.

13. The computer-readable storage medium of claim 12, wherein the process further comprises:
translating, by the interface layer, the output from the one or more on-device machine learning models to JavaScript compatible data, the JavaScript compatible data comprising one or more of an image, a video, audio, text, or a combination thereof; and
performing, by the native JavaScript application, one or more software functions using the JavaScript compatible data.

14. The computer-readable storage medium of claim 12, wherein the on-device runtime environment comprises a C++ runtime environment, the software call comprises one or more JavaScript calls that correspond to one or more C++ library functions, the C++ library functions are registered with an on-device JavaScript runtime environment that executes the native JavaScript application, and the one or more JavaScript calls are translated to one or more calls to the C++ library functions.

15. The computer-readable storage medium of claim 14, wherein executing the software call using the one or more on-device machine learning models comprises executing, via the on-device runtime environment, the translated one or more C++ library function calls using the one or more on-device machine learning models and the data structure representation of the call data.

16. The computer-readable storage medium of claim 12, wherein the software call data comprises an input image or video and the output from the one or more on-device machine learning models comprises an augmented version of the input image or video.

17. The computer-readable storage medium of claim 16, wherein the input image or video is translated, at the translation layer, to a data structure representation of the input image or video, and the on-device machine learning models generate the augmented version of the input image or video using the data structure representation of input image or video.

18. The computer-readable storage medium of claim 17, further comprising:
introspecting a data type for the software call data to determine the input image or video type; and
translating the input image or video type into the data structure representation of the input image or video according to the introspected data type.

19. The computer-readable storage medium of claim 12, further comprising:
introspecting a data type for the output from the one or more on-device machine learning models;
translating, by the interface layer, the output from the one or more on-device machine learning models to JavaScript compatible data according to the introspected data type, the JavaScript compatible data comprising one or more of an image, a video, audio, text, or a combination thereof; and
performing, by the native JavaScript application, one or more software functions using the JavaScript compatible data.

20. A computing system for translating application calls for on-device machine learning execution, the computing system comprising:
one or more processors; and
one or more memories storing instructions that, when executed by the one or more processors, cause the computing system to perform a process comprising:
receiving, from a native JavaScript application, a software call that includes software call data comprising one or more of an image, a video, audio, text, or a combination thereof;
translating, by an interface layer, the software call data to translated call data compatible with one or more on-device machine learning models, wherein the translated call data comprises a data structure representation of the software call data, and the data structure representation comprises a tensor representation, an array or list representation, a dictionary or map representation, a tuple representation, or any combination thereof; and
executing, via an on-device runtime environment, the software call using the one or more on-device machine learning models, wherein the executing generates an output from the one or more on-device machine learning models using the data structure representation of the call data.
